(19) United States
(12) Patent Application Publication
(10) Pub. No.: US 2025/0213445 A1
(43) Pub. Date: Jul. 3, 2025

(54) COMPOSITIONS FOR APPLICATION TO A SURFACE OF SKIN

(71) Applicant: Kao USA Inc., Cincinnati, OH (US)

(72) Inventors: Lauren Carratello, Morrow, OH (US); Eliza Caroline Shook Wilder, Cincinnati, OH (US)

(21) Appl. No.: 18/937,461

(22) Filed: Nov. 5, 2024

Related U.S. Application Data
(60) Provisional application No. 63/615,391, filed on Dec. 28, 2023.

(51) Int. Cl. A61K 8/365; A61K 8/02; A61K 8/73; A61Q 19/00

(52) U.S. Cl. CPC A61K 8/365; A61K 8/0241; A61K 8/731; A61Q 19/00; A61K 2800/412; A61K 2800/413

(57) ABSTRACT

A composition, for application to a surface of skin, includes a first cellulose having a bulk density ranging from 90 g/L to 200 g/L. The composition also includes a second cellulose having a bulk density ranging from 250 g/L to 600 g/L. A ratio of the first cellulose to the second cellulose ranges from 2:1 to 10:1. Additionally, the composition includes from 4.5% to 12% of an alpha-hydroxy acid by weight of the composition.

### CROSS-REFERENCE TO RELATED APPLICATIONS

The present application claims the priority benefit of U.S. Provisional Patent App. Ser. No. 63/615,391, filed Dec. 28, 2023, which is hereby incorporated by reference in its entirety.

### FIELD OF TECHNOLOGY

Disclosed are compositions for application to a surface of skin, as well as methods of making and using the same.

### BACKGROUND

A person's appearance is typically of great importance. Skin conditions, including (but not limited to) acne, eczema, psoriasis, blemishes, and dry skin can cause anxiety and embarrassment to a person. Products exist to treat such skin conditions, often in the form of topical treatments such as gommages for desquamation.

While such products may successfully treat a skin condition, users may experience issues such as discomfort of the skin during treatment, chemical harshness from ingredients in a product, dryness of the skin after treatment, and even allergic reactions.

A need exists for a product that improves or successfully treats a skin condition without creating discomfort, chemical harshness, dryness to the skin, or allergic reactions. The instant disclosure aims to address one or more of the aforementioned needs in the art.

### SUMMARY

Disclosed are compositions for application to a surface of skin, as well as methods of making and using the same. The disclosed compositions may include a dispersant, a first cellulose having a relatively lower bulk density, a second cellulose having a relatively higher bulk density, and an alpha-hydroxy acid. In certain embodiments, the first cellulose can have a bulk density from 90 g/L to 200 g/L and the second cellulose can have a bulk density from 250 g/L to 600 g/L. A ratio of the first cellulose to the second cellulose may range from 2:1 to 10:1. The disclosed compositions may also include from 4.5% to 12% of the alpha-hydroxy acid by weight of the composition.

The disclosed compositions may be used to exfoliate a surface of skin (for instance, a user's face) without a high content of fruit extracts and/or fruit enzymes. The disclosed compositions may do so gently, i.e., without chemical harshness to the surface of skin, while allowing for a soft, smoothened feel to the surface of skin.

### DETAILED DESCRIPTION

A composition for application to a surface of skin is presented. In certain aspects, the composition may include a dispersant, a first cellulose, a second cellulose, and an alpha-hydroxy acid.

### Dispersant

In one aspect, the composition may include a dispersant for carrying and subsuming the remaining ingredients of the composition. The dispersant may be present in an amount necessary to total a weight of the composition to 100% (QS). For instance, the dispersant may be present in an amount ranging from 45% to 90%, or from 50% to 85%, or from 55% to 80%, or from 60% to 75%, or from 65% to 70% by weight of the composition. The dispersant may be present in an amount ranging from 45%, or 50%, or 55%, or 60%, or 65% to 70%, or 75%, or 80%, or 85%, or 90% by weight of the composition. In one aspect, the dispersant is water, and may be deionized, distilled, or purified water.

### First Cellulose

The composition may include a first cellulose. The first cellulose may have a bulk density ranging from 90 g/L to 200 g/L, or from 100 g/L to 175 g/L, or from 105 g/L to 150 g/L, or from 110 g/L to 145 g/L. The bulk density may range from 90 g/L, or 100 g/L, or 105 g/L, or 110 g/L to 145 g/L, or 150 g/L, or 175 g/L, or 200 g/L. In certain versions, the first cellulose can have a particle size ranging from 15 μm to 300 μm with an average particle size ranging from 50 μm to 150 μm. For example, in certain versions, suitable particles of the first cellulose can have a particle size ranging from 25 μm to 225 μm, with an average particle size of 95 μm. Additionally, the first cellulose can have any suitable particle size distribution. For example, the first cellulose can have a particle size of a normal (i.e., Gaussian) distribution having a standard deviation ranging from 25 μm to 250 μm in various versions. In certain versions, the first cellulose can have a particle size ranging from 25 μm to 225 μm, an average particle size of 95 μm, and a standard deviation of up to 110 μm, or a standard deviation averaging 90 μm. In certain versions, 10% of the first cellulose could pass through a 25 μm to 35 μm screen, 50% of the first cellulose could pass through a 55 μm to 80 μm screen, and 90% of the first cellulose could pass through a 125 μm to 250 μm screen. In certain versions, the first cellulose can be commercially obtained. For example, a suitable first cellulose can include Vitacel® CS 200 FC marketed by J. Rettenmaier USA LP.

The first cellulose may be present in the composition in an amount ranging from 5% to 20%, or from 6% to 18%, or from 7% to 15%, or from 8% to 13%, or from 9.5% to 12% by weight of the composition. The first cellulose may be present in an amount ranging from 5%, or 6%, or 7%, or 8%, or 9.5% to 12%, or 13%, or 15%, or 18%, or 20% by weight of the composition. Without intending to be bound by theory, it is believed that the first cellulose has a fluffy texture and can act as an exfoliant to create a “balling effect” of the composition when applied to the surface of skin. The term “balling effect” refers to a clumping or coagulation of the composition while being rubbed into the surface of skin. Without intending to be bound by theory, it is believed that during such clumping or coagulation, the surface of skin is exfoliated, i.e., dead skin cells and debris are collected and removed from the surface of skin.

### Second Cellulose

The composition may include a second cellulose. The second cellulose may have a bulk density ranging from 250 g/L to 600 g/L, or from 275 g/L to 550 g/L, or from 290 g/L to 525 g/L, or from 300 g/L to 500 g/L. The bulk density may range from 250 g/L, or 275 g/L, or 290 g/L, or 300 g/L to 500 g/L, or 525 g/L, or 550 g/L, or 600 g/L. The second cellulose may have a particle size ranging from 90 μm to 300 μm, or from 95 μm to 250 μm, or from 100 μm to 200 μm. The particle size may range from 90 μm, or 95 μm, or 100 μm to 200 μm, or 250 μm, or 300 μm.

The second cellulose may be present in an amount ranging from 0.5% to 4%, or from 1% to 3%, or from 1.2% to 2.5%, or from 1.5% to 2.2%, or from 1.7% to 2.1% by weight of the composition. The second cellulose may be present in an amount ranging from 0.5%, or 1%, or 1.2%, or 1.5%, or 1.7% to 2.1%, or 2.2%, or 2.5%, or 3%, or 4% by weight of the composition. Without intending to be bound by theory, it is believed that the second cellulose has a fine, gritty texture and acts as an exfoliant to smoothen the surface of skin. In certain versions, the second cellulose can be commercially obtained. For example, a suitable second cellulose can include Vitacel® CS 180 G marketed by J. Rettenmaier & Söhne GMBH+CO KG.

In certain versions, the first cellulose may have a relatively lower bulk density (i.e., lower as compared to that of the second cellulose), and the second cellulose may have a relatively higher bulk density (i.e., higher as compared to that of the first cellulose).

### Ratio of First Cellulose to Second Cellulose

In one aspect, a ratio of the first cellulose to the second cellulose in the present compositions may range from 2:1 to 10:1, or from 3:1 to 7.5:1, or from 4:1 to 6:1. The ratio may range from 2:1, or 3:1, or 4:1 to 6:1, or 7.5:1, or 10:1. For instance, the ratio may be 2:1, 3:1, 4:1, 5:1, 6:1, 7:1, 7.5:1, 8:1, 9:1, or 10:1.

### Alpha-Hydroxy Acid

The compositions described herein may include an alpha-hydroxy acid. The alpha-hydroxy acid may be present in an amount ranging from 4.5% to 12%, or from 4.8% to 10%, or from 5% to 8%, or from 5.2% to 7%, or from 5.4% to 6%, by weight of the composition. The amount may range from 4.5%, or 4.8%, or 5%, or 5.2%, or 5.4% to 6%, or 7%, or 8%, or 10%, or 12% by weight of the composition.

Alternatively, the alpha-hydroxy acid may be present in an amount ranging from 0.1% to 25%, or from 0.1% to 12%, or from 0.1% to 10%, by weight of the composition. The amount may range from 0.1% to 25%, or from 1% to 20%, or from 2% to 15%, or from 5% to 10%, or from 6% to 8% by weight of the composition. The amount may range from 0.1%, or 1%, or 2%, or 5%, or 6% to 8%, or 10%, or 15%, or 20%, or 25% by weight of the composition. The amount may range from 0.1% to 12%, or from 1% to 10%, or from 2% to 8%, or from 4% to 6% by weight of the composition. The amount may range from 0.1%, or 1%, or 2%, or 4% to 6%, or 8%, or 10%, or 12% by weight of the composition. The amount may range from 0.1% to 10%, or from 1% to 8%, or from 2% to 7%, or from 4% to 6% by weight of the composition. The amount may range from 0.1%, or 1%, or 2%, or 4%, to 6%, or 7%, or 8%, or 10% by weight of the composition.

In certain versions, the alpha-hydroxy acid may be supplied in an aqueous form to facilitate inclusion into the composition. In other versions, the alpha-hydroxy acid can be supplied as a solid or a dispersed phase within a non-aqueous medium and subsequently dissolved within the composition. Non-limiting examples of suitable alpha-hydroxy acids may include: lactic acid, glycolic acid, malic acid, mandelic acid, phytic acid, azelaic acid, and tartaric acid.

Without intending to be bound by theory, it is believed that the alpha-hydroxy acid may desquamate and smoothen the surface of skin via chemical exfoliation. This may be particularly advantageous, for instance, for a user with dry skin.

In certain versions, the alpha-hydroxy acid can be selected based on particular skin types to be treated. For example, specific alpha-hydroxy acids can be used to treat one or more of dry skin, oily skin, cracked skin, sun-burnt skin, calloused skin, and acne-infested skin. In certain versions for treating dry skin, a suitable alpha-hydroxy acid can be lactic acid. In certain versions, more than one alpha-hydroxy acid can be included.

The composition may include a combined amount of the first cellulose, the second cellulose, and the alpha-hydroxy acid ranging from 10% to 36%, or from 12% to 30%, or from 15% to 25%, or from 16% to 20%, or from 17% to 19% by weight of the composition. The combined amount may range from 10%, or 12%, or 15%, or 16%, or 17% to 19%, or 20%, or 25%, or 30%, or 36% by weight of the composition.

In one version, a ratio of the first cellulose to the alpha-hydroxy acid may range from 0.3:1 to 5:1, or from 5:12 to 4.5:1, or from 0.75:1 to 4:1, or from 0.8:1 to 3.5:1, or from 0.85:1 to 3:1, or from 0.9:1 to 2.5:1, or from 1:1 to 2:1, or from 1.5:1 to 1.8:1. The ratio may range from 0.3:1, or 5:12, or 0.75:1, or 0.8:1, or 0.85:1, or 0.9:1, or 1:1, or 1.5:1 to 1.8:1, or 2:1, or 2.5:1, or 3:1, or 3.5:1, or 4:1, or 4.5:1, or 5:1. For instance, the ratio may be 0.3:1, or 0.5:1, or 0.6:1, or 0.75:1, or 0.8:1, or 5:6, or 6:7, or 7:8, or 1:1, or 10:9, or 1.5:1, or 25:16, or 5:3, or 20:13, or 200:113, or 13:7, or 2:1, or 2.5:1, or 8:3, or 3:1, or 45:13, or 3.5:1, or 4:1, or 40:9, or 4.5:1, or 5:1.

In one version, a ratio of the second cellulose to the alpha-hydroxy acid may range from 0.04:1 to 1:1, or from 1:24 to 8:9, or from 0.05:1 to 0.85:1, or from 0.1:1 to 0.8:1, or from 0.15:1 to 0.75:1, or from 0.2:1 to 0.6:1, or from 0.25:1 to 0.5:1, or from 0.3:1 to 0.4:1. The ratio may range from 0.04:1, or 1:24, or 0.05:1, or 0.1:1, or 0.15:1, or 0.2:1, or 0.25:1, or 0.3 to 0.4:1, or 0.5:1, or 0.6:1, or 0.75:1, or 0.8:1, or 0.85:1, or 8:9, or 1:1. For instance, the ratio may be 0.04:1, or 1:24, or 0.05:1, or 0.1:1, or 0.15:1, or 0.2:1, or 0.25:1, or 0.3:1, or 5:16, or 0.35:1, or 40:113, or 0.4:1, or 0.45:1, or 0.5:1, or 0.55:1, or 0.6:1, or 0.65:1, or 0.7:1, or 0.75:1, or 0.8:1, or 0.85:1, or 8:9, or 0.9:1, or 0.95:1, or 1:1.

### Other Ingredients

The composition may also include other ingredients in addition to the above. Such ingredients may have functions such as (but not limited to): increasing viscosity of the composition (i.e., thickening agent), gelling agent, humectant, moisturizing a surface of skin, anti-microbial agent (i.e., preservative), chelating, prebiotic, emollient, skin conditioning agent, occlusive agent, surfactant, emulsifier, cleansing agent (for cleansing a surface of skin), pH adjuster (for neutralizing, increasing, or decreasing pH of the composition), botanical, and fragrance. Such ingredients may be present in the composition in a solid form or in aqueous form (to facilitate inclusion into the composition).

In one version, the composition may include hydroxyethyl acrylate/sodium acryloyldimethyl taurate copolymer and polyisobutene and PEG-7 trimethylolpropane coconut ether, in an amount ranging from 0.35% to 3%, or from 0.75% to 2.5%, or from 1% to 2% by weight of the composition. The amount may range from 0.35%, or 0.75%, or 1% to 2%, or 2.5%, or 3% by weight of the composition.

In one version, the composition may include glycerin in an amount ranging from 2.5% to 20%, or from 5% to 15%, or from 7.5% to 13% by weight of the composition. The amount may range from 2.5%, or 5%, or 7.5% to 13%, or 15%, or 20% by weight of the composition.

In one version, the composition may include xanthan gum in an amount ranging from 0.05% to 0.5%, or from 0.1% to 0.4%, or from 0.15% to 0.3% by weight of the composition. The amount may range from 0.05%, or 0.1%, or 0.15% to 0.3%, or 0.4%, or 0.5% by weight of the composition.

In one version, the composition may include sodium benzoate in an amount ranging from 0.1% to 1%, or from 0.2% to 0.8%, or from 0.3% to 0.6% by weight of the composition. The amount may range from 0.1%, or 0.2%, or 0.3% to 0.6%, or 0.8%, or 1% by weight of the composition.

In one version, the composition may include tetrasodium glutamate diacetate in an amount ranging from 0.02% to 0.2%, or from 0.05% to 0.15%, or from 0.07% to 0.13% by weight of the composition. The amount may range from 0.02%, or 0.05%, or 0.07% to 0.13%, or 0.15%, or 0.2% by weight of the composition.

In one version, the composition may include inulin in an amount ranging from 0.02% to 0.2%, or from 0.05% to 0.15%, or from 0.07% to 0.13% by weight of the composition. The amount may range from 0.02%, or 0.05%, or 0.07% to 0.13%, or 0.15%, or 0.2% by weight of the composition.

In one version, the composition may include caprylic/capric triglyceride in an amount ranging from 0.1% to 1%, or from 0.25% to 0.75%, or from 0.35% to 0.65% by weight of the composition. The amount may range from 0.1%, or 0.25%, or 0.35% to 0.65%, or 0.75%, or 1% by weight of the composition.

In one version, the composition may include Cocos nucifera (coconut) water and Leuconostoc/radish root ferment filtrate in an amount ranging from 0.002% to 0.02%, or from 0.005% to 0.015%, or from 0.007% to 0.013% by weight of the composition. The amount may range from 0.002%, or 0.005%, or 0.007% to 0.013%, or 0.015%, or 0.02% by weight of the composition.

In one version, the composition may include avocado oil in an amount ranging from 0.02% to 0.2%, or from 0.05% to 0.15%, or from 0.07% to 0.13% by weight of the composition. The amount may range from 0.02%, or 0.05%, or 0.07% to 0.13%, or 0.15%, or 0.2% by weight of the composition.

In one version, the composition may include sodium hydroxide in an amount ranging from 0.3% to 2.5%, or from 0.6% to 2%, or from 0.9% to 1.6% by weight of the composition. The amount may range from 0.3%, or 0.6% or 0.9% to 1.6%, or 2%, or 2.5% by weight of the composition.

In one version, the composition includes less than 3%, or less than 2.5%, or less than 2%, or less than 1%, or less than 0.5%, or less than 0.2%, or less than 0.15% of fruit extracts and/or fruit enzymes by weight of the composition.

The composition may include combinations of the above ingredients listed in this section.

### Exemplary Formulations

The following are exemplary, non-limiting formulations of the composition as disclosed herein. Percentages shown are weight percentages of the total composition, unless otherwise stated:

Example 1: deionized water (QS), hydroxyethyl acrylate/sodium acryloyldimethyl taurate copolymer & polyisobutene & PEG-7 trimethylolpropane coconut ether (2%), glycerin (10%), xanthan gum (0.2%), sodium benzoate (0.4%), tetrasodium glutamate diacetate (0.1%), inulin (0.1%), caprylic/capric triglyceride (0.5%), Cocos nucifera (coconut) water and Leuconostoc/radish root ferment filtrate (0.01%), avocado oil (0.1%), first cellulose (10%), second cellulose (2%), lactic acid (10%), sodium hydroxide (2%).

Example 2: deionized water (QS), hydroxyethyl acrylate/sodium acryloyldimethyl taurate copolymer & polyisobutene & PEG-7 trimethylolpropane coconut ether (1.5%), glycerin (10%), xanthan gum (0.2%), sodium benzoate (0.4%), tetrasodium glutamate diacetate (0.1%), inulin (0.1%), caprylic/capric triglyceride (0.5%), Cocos nucifera (coconut) water and Leuconostoc/radish root ferment filtrate (0.01%), avocado oil (0.1%), first cellulose (10%), second cellulose (2%), lactic acid (2.5%), sodium hydroxide (0.6%).

Example 3: deionized water (QS), hydroxyethyl acrylate/sodium acryloyldimethyl taurate copolymer & polyisobutene & PEG-7 trimethylolpropane coconut ether (1.5%), glycerin (10%), xanthan gum (0.2%), sodium benzoate (0.4%), tetrasodium glutamate diacetate (0.1%), inulin (0.1%), caprylic/capric triglyceride (0.5%), Cocos nucifera (coconut) water and Leuconostoc/radish root ferment filtrate (0.01%), avocado oil (0.1%), first cellulose (15%), second cellulose (3%), lactic acid (5%), sodium hydroxide (1.2%).

Example 4: deionized water (QS), hydroxyethyl acrylate/sodium acryloyldimethyl taurate copolymer & polyisobutene & PEG-7 trimethylolpropane coconut ether (1.5%), glycerin (10%), xanthan gum (0.2%), sodium benzoate (0.4%), tetrasodium glutamate diacetate (0.1%), inulin (0.1%), caprylic/capric triglyceride (0.5%), Cocos nucifera (coconut) water and Leuconostoc/radish root ferment filtrate (0.01%), avocado oil (0.1%), first cellulose (12.5%), second cellulose (2.5%), lactic acid (5%), sodium hydroxide (1.2%).

Example 5: deionized water (QS), hydroxyethyl acrylate/sodium acryloyldimethyl taurate copolymer & polyisobutene & PEG-7 trimethylolpropane coconut ether (1.75%), glycerin (10%), xanthan gum (0.2%), sodium benzoate (0.4%), tetrasodium glutamate diacetate (0.1%), inulin (0.1%), caprylic/capric triglyceride (0.5%), Cocos nucifera (coconut) water and Leuconostoc/radish root ferment filtrate (0.01%), avocado oil (0.1%), first cellulose (12.5%), second cellulose (2.5%), lactic acid (8%), sodium hydroxide (1.8%).

### Method of Making

The following is an exemplary, non-limiting method of making the composition as described herein:

Add hydroxyethyl acrylate/sodium acryloyldimethyl taurate copolymer and polyisobutene and PEG-7 trimethylolpropane coconut ether to water in a main mixing vessel (i.e., a main vessel for mixing) while mixing at low speed to form a first mixture. As thickening occurs, increase mixing speed as needed.

Separately, add xanthan gum to glycerin and mix to uniformity, forming a second mixture. Then add the second mixture to the first mixture and mix to uniformity, forming a third mixture.

To the third mixture, add one at a time of: sodium benzoate, tetrasodium glutamate diacetate, inulin, caprylic/capric triglyceride, Cocos nucifera (coconut) water and Leuconostoc/radish root ferment filtrate, and avocado oil. Continue mixing to uniformity throughout each addition, forming a fourth mixture.

While continuing to mix, add the first cellulose to the fourth mixture. Then add the second cellulose, continuing mixing, forming a fifth mixture. As thickening occurs, increase mixing speed as necessary, while avoiding excess inclusion of air (use side sweep if possible).

Slowly add the alpha-hydroxy acid to the fifth mixture, continuing to mix, to form a sixth mixture.

Separately, add sodium hydroxide to water, forming a seventh mixture. Then add the seventh mixture to the sixth mixture slowly while mixing to form a final mixture (the composition).

As can be appreciated, additional methods of making the composition described herein can also be suitable.

### Method of Use

The following are general, non-limiting methods of use of the composition as disclosed herein:

An amount of the composition (for instance, 1 gram to 3 grams, or the size of a quarter (e.g., a disc that may be 25 mm in diameter)) is dispensed (for instance, squeezed, scooped, or pumped) from a container (for instance, a tube, a jar, or a bottle) onto a hand of a user. The composition is then applied to a surface of skin, for instance to a face, the surface of skin having been previously cleaned and dried. The composition is then rubbed around (for instance, in a circular motion) on the surface of skin, during which the composition undergoes a “balling effect,” as described above. The composition is then rinsed off the surface of skin. A user may apply the composition to the surface of skin multiple times a week, for instance, 2 to 3 times a week.

In one version, the composition is dispensed from a jar by scooping via an applicator such as a stick, swab, wand, or the like. The applicator may be provided separately or be part of the jar (i.e., attached to the jar when initially purchased). For instance, the applicator may be fitted into a lid of the jar (for instance, via a cut-out of the lid), to be slid out from the lid by the user. The applicator allows for a clean dispensing of the composition from a jar (i.e., a user is less likely to contaminate the remaining composition in the jar). The composition can then be deposited onto a hand of the user, and then applied to a surface of skin as detailed above. When finished, the user may slide the applicator back into the lid, possibly after cleaning and drying.

All percentages and ratios are calculated by weight unless otherwise indicated.

All percentages and ratios are calculated based on the total weight of the composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Percent ranges of amounts, ratios, and other numerical values, as disclosed herein, are understood to include the recited value+/−1% unless otherwise specified.

As used herein, articles such as “a” and “an” when used in a claim, are understood to mean one or more of what is claimed or described.

As used herein, the terms “comprising,” “comprises,” “include,” “includes,” and “including” are meant to be non-limiting. The terms “consisting of” or “consisting essentially of” are meant to be limiting, i.e., excluding any components or ingredients that are not specifically listed except when they are present as impurities.

All references, including patent applications, patent publications, and non-patent literature, that are referred to in the present specification are incorporated by reference herein, unless it is expressly indicated that they are not incorporated by reference herein.

Having shown and described various aspects of the present invention, further adaptations of the compositions and methods described herein may be accomplished by appropriate modifications by one of ordinary skill in the art without departing from the scope of the present invention. Several of such potential modifications have been mentioned, and others will be apparent to those skilled in the art. For instance, the examples, materials, ratios, steps, and the like discussed above are illustrative and are not required. Accordingly, the scope of the present invention should be considered in terms of any claims that may be presented and is understood not to be limited to the details of composition and operation described in the specification.

What is claimed is:

1. A composition, for application to a surface of skin, comprising:
a dispersant;
a first cellulose having a bulk density ranging from 90 g/L to 200 g/L;
a second cellulose having a bulk density ranging from 250 g/L to 600 g/L; and
from 4.5% to 12% of an alpha-hydroxy acid by weight of said composition;
said first cellulose and said second cellulose being present in a ratio ranging from 2:1 to 10:1.

2. Said composition of claim 1, wherein said ratio of said first cellulose to said second cellulose ranges from 3:1 to 7.5:1.

3. Said composition of claim 2, wherein said ratio of said first cellulose to said second cellulose ranges from 4:1 to 6:1.

4. Said composition of claim 1, wherein said first cellulose has a bulk density ranging from 100 g/L to 175 g/L.

5. Said composition of claim 4, wherein said first cellulose has a bulk density ranging from 105 g/L to 150 g/L.

6. Said composition of claim 1, wherein said first cellulose has a particle size ranging from 25 μm to 225 μm, and an average particle size of 95 μm.

7. Said composition of claim 1, comprising from 5% to 20% of said first cellulose by weight of said composition.

8. Said composition of claim 1, wherein said second cellulose has a bulk density ranging from 275 g/L to 550 g/L.

9. Said composition of claim 8, wherein said second cellulose has a bulk density ranging from 290 g/L to 525 g/L.

10. Said composition of claim 1, comprising from 0.5% to 4% of said second cellulose by weight of said composition.

11. A composition, for application to a surface of skin, comprising:
a dispersant;
a first cellulose having a bulk density ranging from 100 g/L to 175 g/L;
a second cellulose having a bulk density ranging from 275 g/L to 550 g/L; and
from 4.8% to 10% of an alpha-hydroxy acid by weight of said composition;
said first cellulose and said cellulose being present in a ratio ranging from 3:1 to 7.5:1.

12. Said composition of claim 11, wherein said ratio of said first cellulose to said second cellulose ranges from 4:1 to 6:1.

13. Said composition of claim 11, wherein said first cellulose has a bulk density ranging from 105 g/L to 150 g/L.

14. Said composition of claim 11, comprising from 5% to 20% of said first cellulose by weight of said composition.

15. Said composition of claim 1, wherein said second cellulose has a bulk density ranging from 290 g/L to 525 g/L.

16. Said composition of claim 11, comprising from 0.5% to 4% of said second cellulose by weight of said composition.

17. Said composition of claim 11, comprising from 5% to 8% of said alpha-hydroxy acid by weight of said composition;
wherein said alpha-hydroxy acid is lactic acid.

18. A composition, for application to a surface of skin, comprising:
a dispersant;
a first cellulose having a bulk density ranging from 105 g/L to 150 g/L;
a second cellulose having a bulk density ranging from 290 g/L to 525 g/L; and
from 5% to 8% of an alpha-hydroxy acid by weight of said composition;
said first cellulose and said second cellulose being present in a ratio ranging from 4:1 to 6:1.

19. Said composition of claim 18, comprising from 5% to 20% of said first cellulose by weight of said composition.

20. Said composition of claim 18, comprising from 0.5% to 4% of said second cellulose by weight of said composition.

21. Said composition of claim 20, comprising a combined amount of said first cellulose, said second cellulose, and said alpha-hydroxy acid ranging from 10% to 36% by weight of said composition;
wherein said alpha-hydroxy acid is lactic acid.
